(19) United States
(12) Patent Application Publication
(10) Pub. No.: US 2025/0308662 A1
(43) Pub. Date: Oct. 2, 2025

(54) METHOD AND SYSTEM FOR BRAIN LIQUID BIOPSY AND BRAIN TREATMENT METHOD

(71) Applicants: NATIONAL TAIWAN UNIVERSITY, Taipei (TW); Chang Gung Memorial Hospital,Linkou, Taoyuan City (TW)

(72) Inventors: Hao-Li LIU, Taipei (TW); Chiung-Yin HUANG, Taoyuan City (TW); Ko-Ting CHEN, Taoyuan City (TW); Kuo-Chen WEI, Taoyuan City (TW)

(21) Appl. No.: 18/621,607

(22) Filed: Mar. 29, 2024

(51) Int. Cl. G16H 20/10; A61K 45/06; C12Q 1/6883; G01N 33/49

(52) U.S. Cl. CPC G16H 20/10; A61K 45/06; C12Q 1/6883; G01N 33/5094; G01N 33/6863; G01N 33/6896; G01N 2333/4709; G01N 2800/52

(57) ABSTRACT

A method for brain liquid biopsy includes obtaining a baseline sample from a subject before receiving a first focused ultrasound (FUS) treatment; applying the first FUS treatment to the subject to open a blood-brain barrier (BBB) of the subject; obtaining a first plurality of post-treatment samples respectively at different time points after the first FUS treatment; obtaining a first set of concentration data of a biomarker in the baseline sample and the first plurality of post-treatment samples; and obtaining a kinetic characteristic of the biomarker based on the first set of concentration data.

### MATERIAL INCORPORATED-BY-REFERENCE

The Sequence Listing associated with this application is filed in electronic format via EFS-Web and is hereby incorporated by reference into the specification in its entirety. The name of the XML file containing the Sequence Listing is NP-34597-US_SEQ_LIST.xml. The size of the XML file is 5,250 bytes, and the XML file was created on Mar. 25, 2024.

### BACKGROUND

### Field of Invention

The present disclosure relates to methods and systems for brain liquid biopsy using focused ultrasound and a brain treatment method associated with evaluating brain liquid biopsy.

### Description of Related Art

Liquid biopsy is a medical test that involves the analysis of a patient's blood, urine, or other bodily fluids to detect and analyze circulating tumor cells (CTCs), cell-free DNA (cfDNA), exosomes, or other biomolecules that are released from cancerous cells or other disease or lesion condition. This type of biopsy is often used as a non-invasive alternative to traditional tissue biopsies, which require the removal of a sample of tissue from a tumor or a suspected diseased region for analysis. Liquid biopsy can provide a less invasive way to detect the presence of cancer, monitor the progression of the disease, and track treatment response. Liquid biopsy has been particularly useful in the management of advanced or metastatic cancer, where multiple biopsy samples may be difficult or risky to obtain. It is also being researched for its potential use in early cancer or disease detection, although further studies are needed to validate its accuracy and reliability for this purpose.

Liquid biopsy can potentially be used to diagnose brain diseases, such as brain tumors or neurodegenerative diseases. For brain tumors, liquid biopsy can detect the presence of molecules, e.g., tumor-derived DNA, RNA, or proteins, shed into the bloodstream or cerebrospinal fluid. This can help identify the type of tumor, monitor treatment response, and detect the recurrence of the tumor. However, due to the blood-brain barrier, detecting blood biomarkers originating from brain tumors can be challenging. For neurodegenerative diseases, such as Alzheimer's disease or Parkinson's disease, liquid biopsy can potentially detect biomarkers such as amyloid beta or tau proteins in the blood or cerebrospinal fluid. However, more research is needed to determine the accuracy and reliability of these biomarkers as a diagnostic tool. It's noted that liquid biopsy for brain diseases is still an area of active research, and further studies are needed to establish its clinical utility and accuracy.

### SUMMARY

One aspect of the present disclosure provides methods for brain liquid biopsy. The methods include obtaining a baseline sample from a subject before receiving a first focused ultrasound (FUS) treatment; applying the first FUS treatment to the subject to open a blood-brain barrier (BBB) of the subject; obtaining a first plurality of post-treatment samples respectively at different time points after the first FUS treatment; obtaining a first set of concentration data of a biomarker in the baseline sample and the first plurality of post-treatment samples; and obtaining a kinetic characteristic of the biomarker based on the first set of concentration data.

Another aspect of the present disclosure provides systems for brain liquid biopsy using FUS. The systems include an ultrasound apparatus, a detection equipment, and a processor. The ultrasound apparatus is configured to apply a first FUS treatment to a subject. The detection equipment is configured to receive samples of the subject and obtain concentration data of a biomarker in the samples, wherein the samples comprise a baseline sample and a first plurality of post-treatment samples. The processor is configured to obtain at least a kinetic characteristic of the biomarker.

Another aspect of the present disclosure provides detection methods for brain liquid biopsy using FUS. The detection methods include obtaining a plurality of samples from a subject, wherein the subject receives a first FUS treatment, and the samples comprise a baseline sample before the first focused ultrasound treatment and a first plurality of post-treatment samples after the first FUS treatment; obtaining concentration data of a biomarker in the samples; and obtaining a kinetic characteristic of the biomarker.

In some embodiments, the different time points include a first post-treatment sampling time and a second post-treatment sampling time. The first post-treatment sampling time is set before a plateau phase of secretion of the biomarker. The second post-treatment sampling time is set in the plateau phase of the secretion of the biomarker. In some embodiments, a plurality of concentration-time point data after FUS treatment are obtained, and at least two concentration-time point data are used to obtain the kinetic characteristic. In some embodiments, a plurality of concentration time point data are plotted as concentration-time curves or fitted with pharmacokinetic models to obtain additional kinetic characteristics.

In some embodiments, the kinetic characteristic of the biomarker comprises a concentration-time curve, a concentration ratio of two of the different time points, area under the concentration-time curve (AUC), maximum plasma concentration (Cₘₐₓ), time to reach maximum plasma concentration (Tₘₐₓ), volume of distribution (V_{d}), clearance (CL), steady-state concentration, or a combination thereof.

In some embodiments, obtaining the kinetic characteristic of the biomarker comprises fitting a pharmacokinetic model to the first set of concentration data of the biomarker.

In some embodiments, the pharmacokinetic model includes a compartmental model, a non-compartmental model, or a physiologically-based pharmacokinetic (PBPK) model.

In some embodiments, the methods for brain liquid biopsy further include applying a second FUS treatment to the subject to reopen the BBB of the subject; obtaining a second plurality of post-treatment samples respectively at different time points after the second FUS treatment; and obtaining a second set of concentration data of the biomarker in the second plurality of post-treatment samples. Further, the kinetic characteristic of the biomarker is also based on the second set of concentration data.

In some embodiments, an interval between the first FUS treatment and the second FUS treatment is about 0.1 to 24 hours.

In some embodiments, the parameters of the first FUS treatment and the second FUS treatment comprise the regulation of ultrasound frequency, ultrasound pressure level, ultrasound burst sequence design, microbubble administration design, or the like.

In some embodiments, the biomarker is cell-free RNA, cell-free DNA, mRNA, circulating tumor DNA (DNA), plasma DNA, protein, or peptide.

In some embodiments, the biomarker is EGFR cfDNA, tau, or amyloid beta.

In some embodiments, the method further comprises detecting an immune cell subset or a cytokine of the subject.

In some embodiments, the immune cell subset comprises T cells, B cells, CD8 T cells, CD4 helper T cells, NK cells, or regulatory T cells.

Another aspect of the present disclosure provides brain treatment methods. The brain treatment method includes applying a first therapy to a subject having a brain disease, disorder or lesion; applying a first FUS treatment to the subject to open a blood-brain barrier (BBB) of the subject; obtaining a baseline sample before the first FUS treatment and a first plurality of post-treatment samples respectively at different time points after the first FUS treatment; obtaining a first set of concentration data of a biomarker in the baseline sample and the first plurality of post-treatment samples; obtaining a kinetic characteristic of the biomarker based on the first set of concentration data; and performing an evaluation of the first drug treatment, wherein the evaluation comprises the kinetic characteristic of the biomarker.

In some embodiments, the brain treatment method further includes: detecting an immune cell subset or a cytokine of the subject, wherein the evaluation further comprises a level of the immune cell subset or the cytokine.

In some embodiments, the first therapy comprises a drug treatment, a radiation treatment, a surgical treatment, or a combination thereof.

In some embodiments, the brain treatment method further includes: applying a second therapy to the subject after performing the evaluation of the first drug treatment.

### BRIEF DESCRIPTION OF THE DRAWINGS

The invention can be more fully understood by reading the following detailed description of the embodiment, with reference made to the accompanying drawings as follows:

FIG. 1 illustrates a flow diagram of a method for brain liquid biopsy using FUS, according to some embodiments.

FIG. 2 illustrates a flow diagram of a method for brain liquid biopsy using FUS, according to some embodiments.

FIG. 3 illustrates a diagram of a system for brain liquid biopsy using FUS, according to some embodiments.

FIG. 4A shows the comparison of the cfDNA (egfr) in the peripheral blood of subjects before and after BBB opening (BBBO) induced by FUS treatment.

FIG. 4B shows the correlation between the ratios of cfDNA (egfr) content before and after BBB opening and the ultrasonic exposure levels.

FIG. 4C shows the correlation between the ratios of cfDNA (egfr) content of first and second samples after BBB opening and the ultrasonic exposure levels.

FIGS. 5A to 5C show the CD8 T cell contents in the blood samples from an animal experiment.

FIGS. 6A to 6C show the regulatory T cell contents in the blood samples from an animal experiment.

### DETAILED DESCRIPTION

Reference will now be made in detail to the present embodiments of the disclosure, examples of which are illustrated in the accompanying drawings.

In the following description, certain specific details are set forth in order to provide a thorough understanding of various embodiments of the disclosure. However, one skilled in the art will understand that the disclosure may be practiced without these specific details. In other instances, well-known structures associated with electronic components and operations have not been described in detail to avoid unnecessarily obscuring the descriptions of the embodiments of the present disclosure.

Unless the context requires otherwise, throughout the specification and claims that follow, the word “comprise” and variations thereof, such as “comprises” and “comprising,” are to be construed in an open, inclusive sense, that is, as “including, but not limited to.”

The use of ordinals such as first, second, and third does not necessarily imply a ranked sense of order; rather, it may only distinguish between multiple instances of an act or structure.

Using focused ultrasound (FUS) to transiently open the blood-brain barrier (BBB) is an active research area. It has shown promise for improving drug delivery to the brain for treating brain diseases. In theory, if the BBB could be temporarily opened using focused ultrasound, it could potentially allow biomarkers to pass from the brain into the bloodstream, thereby increasing their concentration in the blood sample.

So far, the current liquid biopsy approach for the central nervous system (CNS) is to do the blood sampling before and after the focused ultrasound BBB opening (BBBO) procedure. The concentration gain can be estimated by the ratio of this two-time's measurement, and the concentration gain can be identified. A preliminary research showed that the CNS liquid biopsy technique utilizing the BBB opening mechanism can boost the biomarker concentration to a gain of 2-3. This increase of biomarker concentration gain, although positive, is still limited, and it may potentially improve the sensitivity of liquid biopsy for CNS diseases, but not much.

Focused ultrasound BBB opening is an active mechanism to change the kinetics of the biomarkers secreted in blood circulation. Before BBB opening, the secretion of the biomarkers tends to stabilize at a certain low level. After doing the BBB opening, the concentration can be transiently increased due to the external active BBB opened mechanism. Once the BBB opening reaches its saturation, the secretion of the biomarkers is maintained at a plateau for at least one hour. The tight junction structure of the BBB has been reported to recover gradually, and the increased leakage of biomarkers gradually returns to its baseline. Therefore, during the BBB open period, the dynamics of the biomarkers go through three phases: (1) baseline phase, (2) transient increase phase, and (3) plateau phase. This transient can be approximated to a biased tangent hyperbolic function. It should be noted that the hyperbolic tangent kinetic represents the kinetics of the biomarker secretion dynamics and theoretically has better sensitivity to the secretion than solely identifying the concentration gain. Accordingly, the sensitivity of liquid biopsy to biomarker detection can be improved by using the characteristics related to the dynamics of biomarkers after BBB is opened through FUS treatment.

Conventional liquid biopsies involve detecting the levels of biomarkers at a single time point within a certain period after treatment (e.g., within the half-life of the biomarker). However, the dynamics of the BBB opening affect the detected biomarker levels. Further, the detected biomarker levels are also influenced by various factors, such as the subject's illness, medication, treatment conditions, or the like. In other words, after the BBB has been opened, biomarkers are released from the brain into the periphery, where their concentration in peripheral blood and tissues is dynamic. Therefore, measuring the concentration of biomarkers at a single time point may provide limited information. Additionally, any timing discrepancies in sampling can result in significant variability in the obtained values.

In various embodiments disclosed herein, utilizing kinetic parameters to reflect the dynamic pattern of biomarker changes within the subject's body aims to obtain more information from liquid biopsy results. Thus, this approach enables a more sensitive and accurate reflection of the pathological mechanisms and biological characteristics of brain diseases in the subjects. In some embodiments, when the concentration ratio at two different time points is calculated based on the data at two concentration-time points, the two different time points are both in the transient increase phase during the secretion of the biomarker.

Some embodiments of the present disclosure disclose that after the BBB is opened by FUS treatment, continuous multiple liquid biopsies are performed to calculate the dynamics of biomarkers. In some embodiments, certain descriptive kinetic properties, such as maximum plasma concentration (Cmₐₓ), time to reach maximum plasma concentration (Tₘₐₓ), or steady-state concentration, can be directly obtained from concentration data and the concentration-time curve after the FUS treatment. Other kinetic parameters can be obtained through mathematical calculations, such as volume of distribution (V_{d}) and clearance rate (CL). In some embodiments, using pharmacokinetic mathematical models can help obtain the kinetic parameters of the biomarkers.

In some embodiments, the kinetic characteristics of biomarkers can be used to assess the effectiveness of therapy and guide adjustments in subsequent therapy strategies, such as treatment type, drug dosage, duration, or the like. Brain liquid biopsies involving FUS treatment are conducted after a subject receives therapy, and the kinetic parameters of disease-related biomarkers are obtained for therapy evaluation. In some embodiments, the therapy comprises a drug treatment, a radiation treatment, a surgical treatment, or a combination thereof.

In some embodiments, the kinetic characteristics of biomarkers can be utilized for long-term monitoring of patient health conditions, such as assessing whether a brain tumor shows signs of transitioning from benign to malignant, which can be evaluated based on the dynamics in the concentration of specific biomarkers.

As used herein, “kinetics of the biomarker” refers to the change(s) of the biomarker within the subject's body over time and the related dynamic processes.

In some embodiments, the biomarker is cell-free RNA, cell-free DNA, mRNA, circulating tumor DNA (DNA), plasma DNA, protein, or peptide.

In some embodiments, the biomarker is associated with a disease, a disorder, a condition, or a lesion of the brain.

In some embodiments, the biomarker is a DNA fragment or an mRNA fragment containing EGFR sequence; in other embodiments, the biomarker is a disease-related protein such as tau, or amyloid beta.

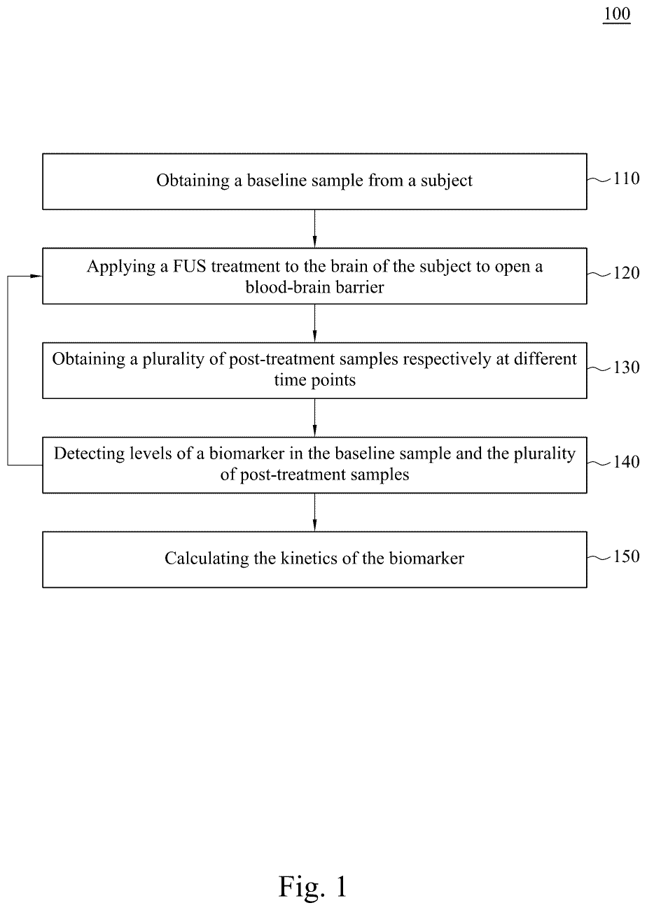

FIG. 1 illustrates the flowchart of a method for brain liquid biopsy using FUS. In step 110 of method 100, a baseline sample is collected from a subject before receiving FUS treatment. In other words, the baseline sample was collected when the subject's BBB was not opened. At least one sample is collected for the baseline of a biomarker. In some embodiments, more than one sample collected at different time points may be used for the baseline of a biomarker.

In step 120 of method 100, the FUS treatment is applied to the subject to open the BBB of the subject. In some embodiments, the FUS treatment is applied at an acoustic pressure and for a period sufficient to disrupt the BBB and release a detectable quantity of a biomarker across BBB. In some embodiments, the method further comprises administering microbubbles to the subject in an amount sufficient to disrupt the BBB upon application of the FUS treatment.

In step 130 of method 100, after the FUS treatment, a plurality of post-treatment samples are collected respectively at different time points. In some embodiments, multiple blood samplings are performed at different time points during the period from 0.1 to 24 hours after the FUS treatment. In some embodiments, the interval between time points of multiple sampling is 1 hour to 2 hours.

In some embodiments, at least one sample is collected at the initial stage when BBB is opened, and at least one sample is collected after the secretion of the biomarker enters the plateau phase.

In step 140 of method 100, a biomarker is detected and quantified in the baseline sample and the post-treatment samples. The different concentrations of the biomarker over time can be obtained.

In some embodiments, the detection method may be or comprise quantification PCR or sequencing detection. The quantification PCR may be digital PCR, such as droplet digital PCR (ddPCR). The sequencing detection may be deep-sequencing technology, such as AmpliSeq, HaloPlex sequencing, or the like.

In step 150 of method 100, the kinetics of the biomarker are calculated. In some embodiments, the kinetic properties of the biomarker comprise a concentration-time curve, a concentration ratio of the different time points, area under the concentration-time curve (AUC), maximum plasma concentration (Cₘₐₓ), time to reach maximum plasma concentration (Tₘₐₓ), volume of distribution (V_{d}), clearance (CL), steady-state concentration, or a combination thereof.

In some embodiments, some of the kinetic parameters of the biomarker are obtained through pharmacokinetic analysis. Pharmacokinetics can measure and explain the change of drug concentration with time in a subject's body after administration. Therefore, the concentration data of the biomarker can be used to obtain kinetic parameters like pharmacokinetic parameters, which can be used to estimate the dynamics of a substance in the body, such as absorption rate, distribution rate, metabolic rate, and elimination rate (ADME).

In some embodiments, estimating the kinetic parameters of the biomarker involves using mathematical models to simulate the ADME of the biomarker in the body based on the concentration data obtained from blood sampling. These mathematical models can be used to estimate key parameters such as the half-life of the biomarker in the blood, the clearance rate from the bloodstream, and the volume of distribution in different tissues.

In some embodiments, the choice of modeling approach depends on the specific biomarker and the disease being studied, as well as the available data and resources. There are many different mathematical models and approximations that have been developed for pharmacokinetic modeling. In some embodiments, the mathematical models include compartmental modeling, non-compartmental modeling, or physiologically-based pharmacokinetic (PBPK) modeling. These mathematical models use different mathematical equations to describe the ADME of the biomarker in the body, and they can be used to estimate various kinetic parameters such as the half-life, clearance rate, and volume of distribution.

In some embodiments, software tools such as Phoenix WinNonlin, NONMEM, or Simcyp are used for pharmacokinetic modeling, allowing researchers to fit different models to experimental data and estimate pharmacokinetic parameters.

Referring to FIG. 1, in some embodiments, optionally, feedback control may be implemented in step 140. The ultrasound exposure levels affect the degree of BBB opening and influence the amounts of the released biomarkers. In some embodiments, feedback control of ultrasound exposure level is based on the detected quantity of biomarkers. In some embodiments, a threshold value is set, and when the biomarker concentration is higher than the threshold value, subsequent sampling, detection, and kinetic analysis are conducted. When the biomarker concentration falls below the threshold value, focused ultrasound is applied again, and samples are retaken to detect biomarker concentrations. Repeated FUS treatment may prolong the opening of BBB or increase the degree of BBB opening. Step 120 may be repeated to apply FUS treatment until the detected value of the biomarker reaches the threshold value.

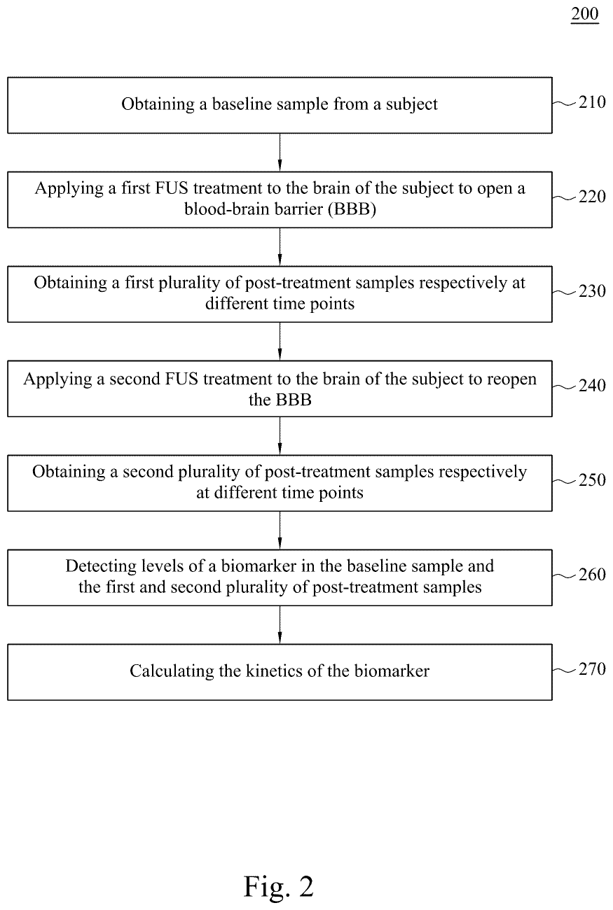

FIG. 2 illustrates the flowchart of a method for brain liquid biopsy using FUS in alternative embodiments. Steps 210-230 of method 200 in FIG. 2 are similar to steps 110-130 of method 100 in FIG. 1. The difference between method 200 and method 100 is applying the second FUS treatment to the subject in method 200. In yet other embodiments, more FUS treatments can be applied to the subject. The kinetic parameters of the biomarker for different rounds can be obtained and compared, which can be used to monitor disease or lesions, treatment response, or treatment efficacy.

In some embodiments, repeated focused ultrasonic treatments to repeatedly open BBB can promote the biomarker to leak more from the brain to the peripheral blood. Therefore, kinetic analysis for the biomarker can be more accurate.

In some embodiments, different rounds of focused ultrasound treatments can be applied to different brain regions of a subject, to detect and analyze the biomarker levels of these different brain regions.

In some embodiments, an interval between the first FUS treatment and the second FUS treatment is about 0.1 to 24 hours.

In some embodiments, parameters of the first FUS treatment and the second FUS treatment comprise the regulation of ultrasound frequency, ultrasound pressure level, ultrasound burst sequence design (combination of pulse repetition frequency, pulse length, and sonication duration), and microbubble administration design (IV bolus or infusion with a controlled given flow rate, venous or artery administration). Optimization of the acoustic parameters (e.g., acoustic pressure, pulse repetition frequency, pulse length, and sonication duration) of the first and second FUS treatments can be performed to enhance biomarker release with minimal or no tissue damage.

In step 240 of method 200, after the second FUS treatment, a plurality of post-treatment samples are collected respectively at different time points.

In step 260 of method 200, the biomarker is detected and quantified in the baseline sample and the first and second post-treatment samples. The concentration of the biomarker over time and a related curve can be obtained.

In step 270 of method 200, the kinetic parameters of the biomarker are calculated. Step 270 of method 200 is similar to step 150 of method 100, except for the kinetic parameters of the biomarker in the subject receiving two FUS treatments.

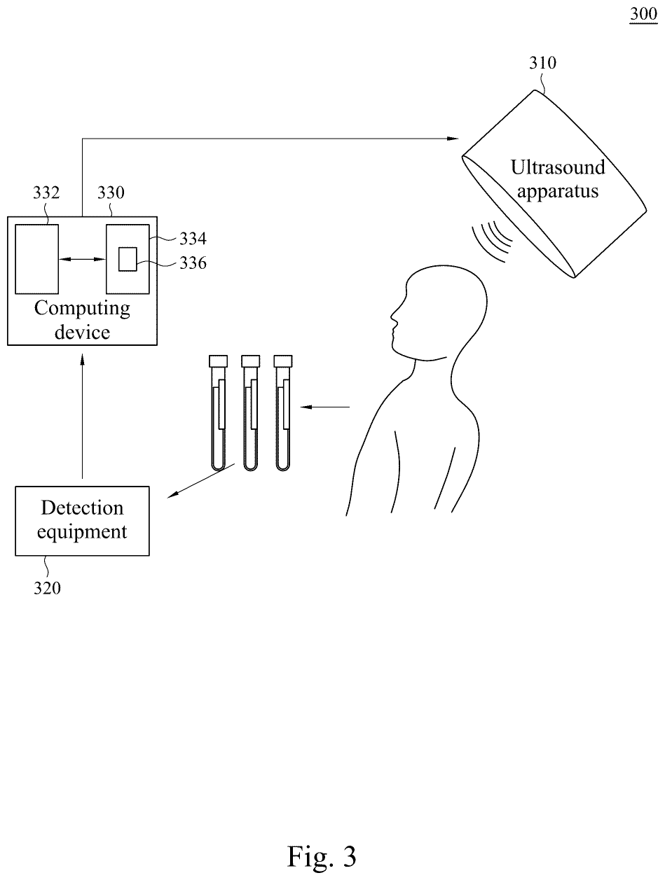

FIG. 3 illustrates a diagram of a system according to some embodiments. System 300 includes an ultrasound apparatus 310, a detection equipment 320, and a computing device 330. The ultrasound apparatus 310 comprises an ultrasound transducer configured to emit a focused ultrasound beam to a subject. The detection equipment 320 is configured to receive samples of the subject and obtain concentration data of a biomarker in the samples, wherein the samples comprise a baseline sample before the FUS treatment and a first plurality of post-treatment samples. The computing device 330 is configured to calculate the kinetics of the biomarker.

As shown in FIG. 3, the computing device 330 includes a processor 332 and a storage device 334 which are electrically connected with each other. The storage device 334 includes a non-transient computer-readable medium 336. The non-transient computer-readable medium 336 stores processing programs and mathematical models for calculating the kinetics of the biomarker according to concentration data from the detection equipment 320. The processor 332 performs the following steps: acquiring concentration data over time of a biomarker from a subject receiving FUS treatment, and fitting the mathematical models to the concentration data and estimating the kinetic parameters of the biomarker.

Referring to FIG. 3, in some embodiments, the computing device 330 and the ultrasound apparatus 310 can be communicatively coupled, for example, via a wired network or a wireless network, and the system 300 has the capability of feedback control. When the biomarker concentration after the first FUS treatment is obtained, the pulse repetition frequency, pulse length, sonication duration, or the like of the ultrasound apparatus 310 for the subsequent FUS treatment(s) can be adjusted. In some implementations, system 300 is designed as a closed-loop system with feedback control. FUS treatment is applied, and the biomarker concentration in blood is detected to confirm whether the biomarker concentration reaches a predetermined threshold. If the biomarker concentration does not reach this predetermined threshold, FUS treatment is applied again, and the biomarker concentration in blood is detected again. The processes are repeated until the biomarker concentration reaches the threshold.

In one clinical trial, to validate the FUS-assisted liquid biopsy procedure (also called a sonobiopsy), blood samples were collected from four subjects before and after FUS-BEV treatments, and concentrations and kinetics of cfDNA containing egfr sequence were determined. The blood samples were collected into EDTA-coated tubes (366643, BD, USA) and centrifuged at 2500×g for 10 min at 4° C. The clarified supernatant was aliquoted and then stored at −80° C. before being analyzed. Samples were then prepared for droplet digital PCR (ddPCR). One milliliter of aliquoted plasma was thawed on ice and centrifuged for 10 min at 14,000×g at 4° C. The QIAamp MinElute ccfDNA Kit (55204, Qiagen, Hilden, Germany) was used to extract cell-free DNA (cfDNA) according to the manufacturer's instructions, with the final product dissolved in 60 μl of ultraclean water. The DNA concentration was measured using a bioanalyzer (2100, Agilent, CA, USA). ddPCR was performed using the QuantStudio™ 3D Digital PCR system with the QuantStudio™ 3D Digital PCR Master Mix (version 2) and the QuantStudio™ 3D Digital PCR 20K Chip Kit (version 2).

The epidermal growth factor receptor (EGFR) tumor-specific gene and the hydroxymethylbilane synthase (HMBS) housekeeping gene were analyzed using a method with universal LNA-hydrolysis probes from the 96 Universal Probe Library® (UPL, Sigma-Aldrich®, St. Louis, Missouri, USA). The EGFR primers were 5′-ACCCCACTTTATAGAGAGGTAGACTG-3′ (i.e., SEQ NO.1 in the Sequence Listing) and 5′-AGTCTTGGGCAATTTGCTTC-3′ (i.e., SEQ NO.2 in the Sequence Listing) with UPL® probe #11 (5′-CTTCCAGC-3′), and the HMBS primers were 5′-GGGACAGTGTACCCAAGGTC-3′ (i.e., SEQ NO.3 in the Sequence Listing) and 5′-CTGAGGTAAACGGATCTGACG-3′ (i.e., SEQ NO.4 in the Sequence Listing) with probe 5′-CCAAGAGGCTGAGCAGGACT-3′ (i.e., SEQ NO.5 in the Sequence Listing).

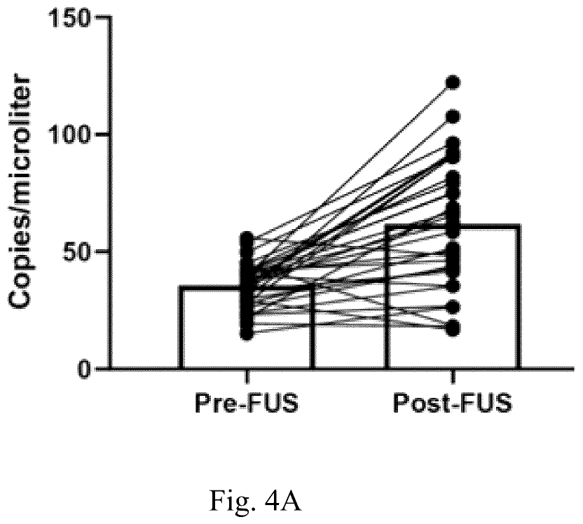

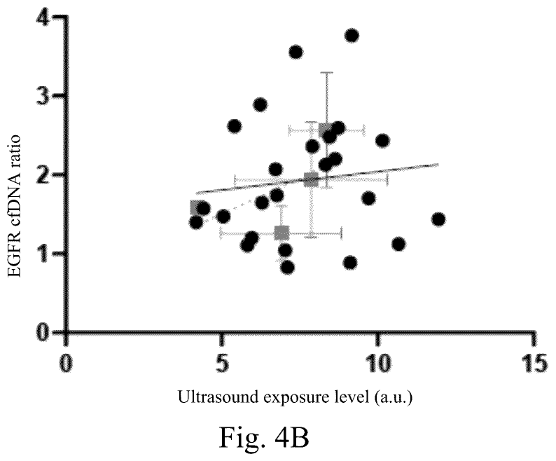

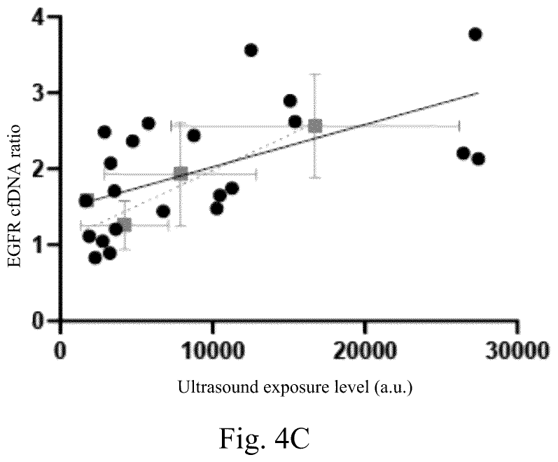

FIGS. 4A-4B show the results of analyzing the baseline concentration of the biomarker before FUS treatment and the biomarker concentration of a sample after FUS treatment. FIG. 4C shows the results of analyzing the biomarker concentration in the first sample and the second sample after the FUS treatment, wherein the sampling interval between the first sample and the sample was 0.5 to 1 hour.

In FIG. 4A, the serum concentrations of EGFR cfDNA before and after FUS were compared (22 paired data). FIG. 4A shows that significant elevations of the concentrations of cfDNA of 1.77±0.76-fold, respectively, were detected in plasma samples (p<0.001 and p<0.0001, respectively), suggesting their usability as liquid surrogates for FUS-BBBO.

FIG. 4B shows that the correlation between the EGFR cfDNA ratio and the ultrasound exposure level. In FIG. 4B, the EGFR cfDNA ratio is the ratio of the EGFR level after FUS treatment to the baseline EGFR level. The range of each gray square is based on the data of a same subject, and r²=0.08. Each black dot is based on the data of each FUS treatment, and r²=0.005. FIG. 4B shows that the correlation between the EGFR ratio and the ultrasound exposure level is low, and r²=0.35.

FIG. 4C shows the correlation between the ratios of cfDNA of first and second samples after BBB opening and the ultrasonic exposure level. In FIG. 4C, The EGFR cfDNA ratio is the ratio of the EGFR level of the first sample to the EGFR level of the second sample. The range of each gray square is based on the data of a same subject. Each black dot is based on the data of each FUS treatment. FIG. 4C shows that the correlation between the EGFR cfDNA ratio after FUS treatment and the ultrasound exposure level is high, and r²=0.84.

In other words, the value of biomarkers in a single sampling after FUS cannot reflect the correlation between biomarkers passing through the BBB and ultrasound exposure level. By contrast, the kinetic characteristic of the biomarkers from multiple sampling after FUS treatment can improve the correlation with the ultrasound exposure level, and the value of r² increases from 0.35 to 0.84.

These findings indicate that (1) FUS-BBBO enhances the concentration of circulating cfDNA and facilitates brain tumor-specific liquid biopsies, (2) the ratio of pre-to post-FUS cfDNA may be a serum biomarker of BBBO, and (3) solely relying on the measured ratio of cfDNA is not sufficient for predicting the BBBO, while combining with the kinetics of ratio can significantly improve the BBBO predictability.

In some embodiments, the method also includes monitoring the subject's disease and medication status, and the biomarker kinetics information obtained by combining FUS treatment and liquid biopsy can help to better understand the pathological mechanism and therapeutic response of brain diseases, disease, or lesion.

Since the FUS-induced BBB opening (FUS-BBBO) may lead to immunoregulatory responses in the brain, in some embodiments, immune cells in peripheral blood or their secreted effectors, such as cytokines, are also detected. In terms of brain tumors or infectious diseases, some specific lymphocyte subtypes or cytokines have been reported to have immunomodulatory effects, so monitoring the immune response during or after FUS-BBBO may be helpful in choosing the appropriate therapies.

In some embodiments, the immune cell to be detected comprises different cell subsets, such as B cells, T cells, NK cells, CD8 T cells, CD4 helper T cells, or regulatory T cells. For brain tumors, such as glioblastoma, regulatory T lymphocyte (Treg) has been reported to play an immune inhibitory role, and CD8+ T cells may act as effectors in the tumor microenvironment. Specifically, it is worthwhile to examine the changes in the ratios of CD8+/Treg cells secreted from the brain tumors.

In some embodiments, a portion of the peripheral blood collected before and after FUS treatment is used to isolate cells, such as peripheral blood mononuclear cells (PMBC), and detect the content of specific lymphocyte subtypes. In some embodiments, the plasma separated from the above peripheral blood can be used to detect the cytokine secreted by immune cells, such as IFN-γ, IL-2, IL-10, IL-12, IL-17, GM-CSF, TGF-TGF-β, TNF-α, or the like.

In the following experiments using rat as a glioma animal model, the changes in cell counts after FUS treatment with two FUS sonication pressure levels (0.63 and 0.8 MI) were analyzed. CD8 T cells were identified as CD3+ and CD8+, and Treg cell was identified as CD4+, CD25+, and Foxp3+. The peripheral blood cells of the rats were stained with antibodies and the contents of specific cells were determined by flow cytometry. Flow cytometry analyses were performed on a three-color fluorescence FACS caliburcytometer using Cell Quest software (Becton-Dickinson, CA, USA).

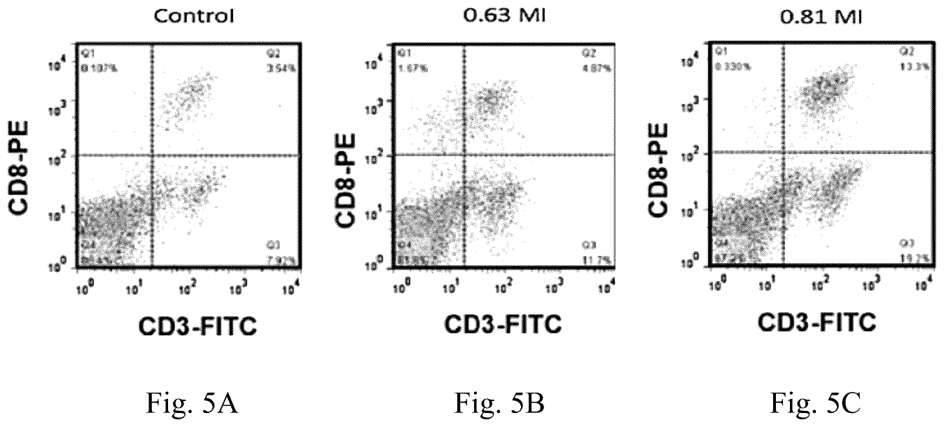

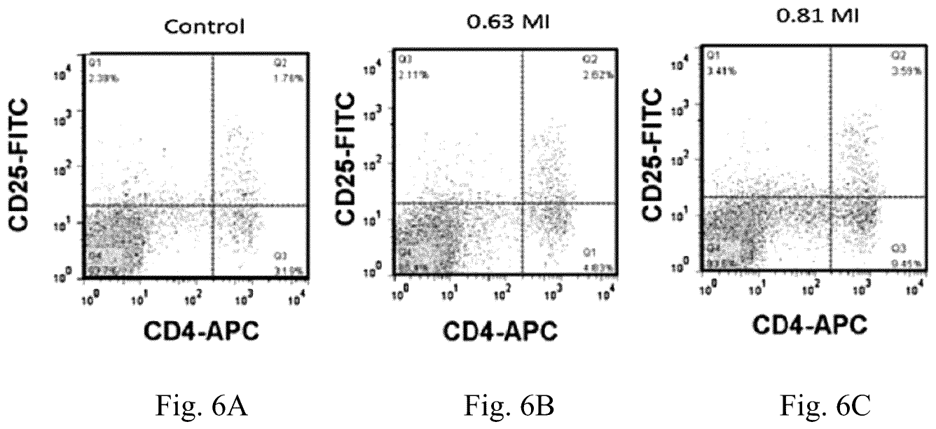

FIGS. 5A to 5C shows that a significant increase in CD8+ T lymphocytes after 0.8-MI FUS treatment in comparison to the untreated controlled animals (P=0.056). In addition, no significant change of Treg (CD4+CD25+Foxp3+) counts after 0.8-MI FUS treatment was found (P=0.072, respectively). FIGS. 6A to 6C show the CD4+CD25+ cells. For 0.63-MI exposure, there was no statistically significant increase for both CD8 T cells and Treg cells.

There was no obvious CD8+/Treg ratio change in 0.63 MI group compared to the control. On the other hand, it was observed that FUS-BBB opening at the proper energy level (0.81 MI) indeed resulted in an increase in the CD8+/Treg ratio when compared with the control group; 1.19±0.38 versus 2.12±0.70; p=0.035), supporting that 0.81-MI BBBO sonication provided the most profound CD8+/Treg ratio increase. It indicates a synergistic effect on immunological changes in the tumor region that are beneficial in the suppression of glioma progression.

Embodiments of the present disclosure provide methods and systems for brain biopsy to obtain the dynamic characteristic(s) of biomarkers after FUS treatment, thus increasing the sensitivity and accuracy of liquid biopsy detection. This allows for understanding the regularity of secretion dynamics of biomarkers after the blood-brain barrier is opened, which is helpful for understanding the pathological mechanism, evaluating the therapeutic response, and selecting the therapeutic scheme of a subject.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims.

What is claimed is:

1. A method for brain liquid biopsy, comprising:
obtaining a baseline sample from a subject before receiving a first focused ultrasound (FUS) treatment;
applying the first FUS treatment to the subject to open a blood-brain barrier (BBB) of the subject;
obtaining a first plurality of post-treatment samples respectively at different time points after the first FUS treatment;
obtaining a first set of concentration data of a biomarker in the baseline sample and the first plurality of post-treatment samples; and
obtaining a kinetic characteristic of the biomarker based on the first set of concentration data.

2. The method for brain liquid biopsy of claim 1, wherein the kinetic characteristic of the biomarker comprises: a concentration-time curve, a concentration ratio of two of the different time points, area under the concentration-time curve (AUC), maximum plasma concentration (Cₘₐₓ), time to reach maximum plasma concentration (Tₘₐₓ), volume of distribution (V_{d}), clearance (CL), steady-state concentration, or a combination thereof.

3. The method for brain liquid biopsy of claim 2, wherein the obtaining the kinetic characteristic of the biomarker comprises fitting a pharmacokinetic model to the first set of concentration data of the biomarker.

4. The method for brain liquid biopsy of claim 3, wherein the pharmacokinetic model comprises: a compartmental model, a non-compartmental model, or a physiologically-based pharmacokinetic (PBPK) model.

5. The method for brain liquid biopsy of claim 1, further comprising:
applying a second FUS treatment to the subject to reopen the BBB of the subject;
obtaining a second plurality of post-treatment samples respectively at different time points after the second FUS treatment; and
obtaining a second set of concentration data of the biomarker in the second plurality of post-treatment samples;
wherein the kinetic characteristic of the biomarker is also based on the second set of concentration data.

6. The method for brain liquid biopsy of claim 5, wherein an interval between the first FUS treatment and the second FUS treatment is about 0.1 to about 24 hours.

7. The method for brain liquid biopsy of claim 1, wherein the biomarker is cell-free RNA, cell-free DNA, mRNA, circulating tumor DNA (DNA), plasma DNA, protein, or peptide.

8. The method for brain liquid biopsy of claim 1, wherein the biomarker is EGFR cfDNA, tau, or amyloid beta.

9. The method for brain liquid biopsy of claim 1, further comprises:
detecting an immune cell subset or a cytokine of the subject.

10. The method for brain liquid biopsy of claim 9, wherein the immune cell subset comprises T cells, B cells, CD8 T cells, CD4 helper T cells, NK cells, or regulatory T cells.

11. A system for brain liquid biopsy, comprising:
an ultrasound apparatus configured for applying a first focused ultrasound (FUS) treatment to a subject;
a detection equipment configured for receiving samples of the subject and obtaining concentration data of a biomarker in the samples, wherein the samples comprise a baseline sample and a first plurality of post-treatment samples; and
a computing device configured for obtaining a kinetic characteristic of the biomarker.

12. The system for brain liquid biopsy of claim 11, wherein the kinetic characteristic of the biomarker comprises: a concentration-time curve, a concentration ratio of different time points, area under the concentration-time curve (AUC), maximum plasma concentration (Cₘₐₓ), time to reach maximum plasma concentration (Tₘₐₓ), volume of distribution (V_{d}), clearance (CL), steady-state concentration, or a combination thereof.

13. The system for brain liquid biopsy of claim 11, wherein the obtaining the kinetic characteristic of the biomarker comprises fitting a pharmacokinetic model to the concentration data of the biomarker.

14. The system for brain liquid biopsy of claim 13, wherein the computing device comprises a non-transient computer-readable medium configured to store the pharmacokinetic model.

15. A brain treatment method comprising:
applying a first therapy to a subject having a brain disease, disorder or lesion;
applying a first focused ultrasound (FUS) treatment to the subject to open a blood-brain barrier (BBB) of the subject;
obtaining a baseline sample before the first FUS treatment and a first plurality of post-treatment samples respectively at different time points after the first FUS treatment;
obtaining a set of concentration data of a biomarker in the baseline sample and the first plurality of post-treatment samples;
obtaining a kinetic characteristic of the biomarker according to the first set of concentration data; and
performing an evaluation of the first therapy, wherein the evaluation comprises the kinetic characteristic of the biomarker.

16. The brain treatment method of claim 15, further comprising:
detecting an immune cell subset or a cytokine of the subject, wherein the evaluation further comprises a level of the immune cell subset or the cytokine.

17. The brain treatment method of claim 15, wherein the first therapy comprises a drug treatment, a radiation treatment, a surgical treatment, or a combination thereof.

18. The brain treatment method of claim 15, further comprising:
applying a second therapy to the subject after performing the evaluation of the first therapy.


SEQUENCE LISTING (5 sequences)

SEQ ID NO: 1 - DNA, 26 bp, synthetic construct
  source 1..26: mol_type = other DNA
accccactttatagagaggtagactg

SEQ ID NO: 2 - DNA, 20 bp, synthetic construct
  source 1..20: mol_type = other DNA
agtcttgggcaatttgcttc

SEQ ID NO: 3 - DNA, 20 bp, synthetic construct
  source 1..20: mol_type = other DNA
gggacagtgtacccaaggtc

SEQ ID NO: 4 - DNA, 21 bp, synthetic construct
  source 1..21: mol_type = other DNA
ctgaggtaaacggatctgacg

SEQ ID NO: 5 - DNA, 20 bp, synthetic construct
  source 1..20: mol_type = other DNA
ccaagaggctgagcaggact